(19) United States
(12) Patent Application Publication
(10) Pub. No.: US 2012/0046754 A1
(43) Pub. Date: Feb. 23, 2012

(54) APPARATUS FOR CONTROLLING A LOAD ON A HIP JOINT

(75) Inventors: Anton G. Clifford, Mountain View, CA (US); Stefan Gabriel, Mattapoisett, MA (US); Michael E. Landry, Austin, TX (US); Mary O'Connell, Menlo Park, CA (US); Josef L. Friedmann, Scotts Valley, CA (US); Michael Rode, Lake Oswego, OR (US)

(73) Assignee: MOXIMED, INC., Hayward, CA (US)

(21) Appl. No.: 13/194,189

(22) Filed: Jul. 29, 2011

Related U.S. Application Data
(60) Provisional application No. 61/374,800, filed on Aug. 18, 2010.

(51) Int. Cl. A61F 2/32

(52) U.S. Cl. 623/22.11

(57) ABSTRACT

An apparatus and related method for controlling a load on a human hip joint during normal gait while preserving motion. The approach is intended to treat osteoarthritis of the hip without substantially resisting an angular displacement associated with full mobility of the pelvis and femur bones.

### REFERENCES TO RELATED APPLICATIONS

This application claims the benefit of U.S. application Ser. No. 61/374,800, filed Aug. 18, 2010, the entire disclosure of which is expressly incorporated herein.

### BACKGROUND OF THE INVENTION

The present disclosure is directed toward apparatus and methods for treating joints and in particular, to treating hip joints affected with osteoarthritis.

A joint is the location at which two or more bones make contact. Joints are constructed to allow movement and provide mechanical support, and are classified structurally and functionally. Structural classification is determined by how the bones connected to each other, while functional classification is determined by the degree of movement between the articulating bones. In practice, there is significant overlap between the two types of classifications.

There are three structural classifications of joints, namely fibrous or immovable joints, cartilaginous joints and synovial joints. Fibrous/Immovable bones are connected by dense connective tissue, consisting mainly of collagen. The fibrous joints are further divided into three types:

sutures which are found between bones of the skull;

syndesmosis which are found between long bones of the body; and

gomphosis which is a joint between the root of a tooth and the sockets in the maxilla or mandible.

Cartilaginous bones are connected entirely by cartilage (also known as “synchondroses”). Cartilaginous joints allow more movement between bones than a fibrous joint but less than the highly mobile synovial joint. Cartilaginous joints include the artificial discs of the spine.

Synovial joints have a space between the articulating bones for synovial fluid. This classification contains joints that are the most mobile of the three, and includes the hip, knee and shoulder. These are further classified into ball and socket joints, condyloid joints, saddle joints, hinge joints, pivot joints, and gliding joints.

Joints can also be classified functionally, by the degree of mobility they allow. Synarthrosis joints permit little or no mobility. They can be categorized by how the two bones are joined together. That is, synchrondoses are joints where the two bones are connected by a piece of cartilage. Synostoses are where two bones that are initially separated eventually fuse together as a child approaches adulthood. By contrast, amphiarthrosis joints permit slight mobility. The two bone surfaces at the joint are both covered in hyaline cartilage and joined by strands of fibrocartilage. Most amphiarthrosis joints are cartilaginous.

Finally, diarthrosis joints permit a variety of movements (e.g. flexion, adduction, pronation). Only synovial joints are diarthrodial and they can be divided into six classes: 1. ball and socket—such as the shoulder or the hip and femur; 2. hinge—such as the elbow; 3. pivot—such as the radius and ulna; 4. condyloidal (or ellipsoidal)—such as the wrist between radius and carps, or knee; 5. saddle—such as the joint between carpal thumbs and metacarpals; and 6. gliding—such as between the carpals.

Synovial joints (or diarthrosis, or diarthroidal joints) are the most common and most moveable type of joints in the body. As with all other joints in the body, synovial joints achieve movement at the point of contact of the articulating bones. Structural and functional differences distinguish the synovial joints from the two other types of joints in the body, with the main structural difference being the existence of a cavity between the articulating bones and the occupation of a fluid in that cavity which aids movement. The whole of a diarthrosis is contained by a ligamentous sac, the joint capsule or articular capsule. The surfaces of the two bones at the joint are covered in cartilage. The thickness of the cartilage varies with each joint, and sometimes may be of uneven thickness. Articular cartilage is multi-layered. A thin superficial layer provides a smooth surface for the two bones to slide against each other. Of all the layers, it has the highest concentration of collagen and the lowest concentration of proteoglycans, making it very resistant to shear stresses. Deeper than that is an intermediate layer, which is mechanically designed to absorb shocks and distribute the load efficiently. The deepest layer is highly calcified, and anchors the articular cartilage to the bone. In joints where the two surfaces do not fit snugly together, a meniscus or multiple folds of fibro-cartilage within the joint correct the fit, ensuring stability and the optimal distribution of load forces. The synovium is a membrane that covers all the non-cartilaginous surfaces within the joint capsule. It secretes synovial fluid into the joint, which nourishes and lubricates the articular cartilage. The synovium is separated from the capsule by a layer of cellular tissue that contains blood vessels and nerves.

Various maladies can affect the joints, one of which is arthritis. Arthritis is a group of conditions where there is damage caused to the joints of the body. Arthritis is the leading cause of disability in people over the age of 65.

There are many forms of arthritis, each of which has a different cause. Rheumatoid arthritis and psoriatic arthritis are autoimmune diseases in which the body is attacking itself. Septic arthritis is caused by joint infection. Gouty arthritis is caused by deposition of uric acid crystals in the joint that results in subsequent inflammation. The most common form of arthritis, osteoarthritis is also known as degenerative joint disease and occurs following trauma to the joint, following an infection of the joint or simply as a result of aging.

Unfortunately, all arthritides feature pain. Patterns of pain differ among the arthritides and the location. Rheumatoid arthritis is generally worse in the morning; in the early stages, patients often do not have symptoms following their morning shower.

Osteoarthritis (OA, also known as degenerative arthritis or degenerative joint disease, and sometimes referred to as “arthrosis” or “osteoarthrosis” or in more colloquial terms “wear and tear”), is a condition in which low-grade inflammation results in pain in the joints, caused by wearing of the cartilage that covers and acts as a cushion inside joints. As the bone surfaces become less well protected by cartilage, the patient experiences pain upon weight bearing, including walking and standing. Due to decreased movement because of the pain, regional muscles may atrophy, and ligaments may become more lax. OA is the most common form of arthritis.

The main symptoms of osteoarthritis is chronic pain, causing loss of mobility and often stiffness. “Pain” is generally described as a sharp ache, or a burning sensation in the associated muscles and tendons. OA can cause a crackling noise (called “crepitus”) when the affected joint is moved or touched, and patients may experience muscle spasm and contractions in the tendons. Occasionally, the joints may also be filled with fluid. Humid weather increases the pain in many patients.

OA commonly affects the hand, feet, spine, and the large weight-bearing joints, such as the hips and knees, although in theory, any joint in the body can be affected. As OA progresses, the affected joints appear larger, are stiff and painful, and usually feel worse, the more they are used and loaded throughout the day, thus distinguishing it from rheumatoid arthritis. With progression in OA, cartilage loses its viscoelastic properties and its ability to absorb load.

Generally speaking, the process of clinical detectable osteoarthritis is irreversible, and typical treatment consists of medication or other interventions that can reduce the pain of OA and thereby improve the function of the joint. According to an article entitled Surgical Approaches for Osteoarthritis by Klaus-Peter Gunther, MD, over recent decades, a variety of surgical procedures have been developed with the aim of decreasing or eliminating pain and improving function in patients with advanced osteoarthritis (OA). The different approaches include preservation or restoration of articular surfaces, total joint replacement with artificial implants, and arthrodesis (fusion).

Arthrodesis are described as being reasonable alternatives for treating OA of small hand and foot joints as well as degenerative disorders of the spine, but were deemed to be rarely indicated in large weight-bearing joints such as the hip due to functional impairment of gait, cosmetic problems and further side-effects. Total joint replacement was characterized as an extremely effective treatment for severe joint disease. Moreover, recently developed joint-preserving treatment modalities were identified as having a potential to stimulate the formation of a new articular surface in the future. However, it was concluded that such techniques do not presently predictably restore a durable articular surface to an osteoarthritic joint. The correction of mechanical abnormalities by osteotomy and joint debridement are still considered as treatment options in many patients.

Joint replacement is one of the most common and successful operations in modern orthopaedic surgery. It consists of replacing painful, arthritic, worn or diseased parts of the joint with artificial surfaces shaped in such a way as to allow joint movement. Such procedures are a last resort treatment as they are highly invasive and require substantial periods of recovery. Joint replacement is sometimes called total joint replacement indicating that all joint surfaces are replaced. This contrasts with hemiarthroplasty (half arthroplasty) in which only one bone's joint surface is replaced and unincompartmental arthroplasty in which both surfaces of the knee, for example, are replaced but only on the inner or outer sides, not both. Thus, arthroplasty as a general term, is an operative procedure of orthopaedic surgery performed, in which the arthritic or dysfunctional joint surface is replaced with something better or by remodeling or realigning the joint by osteotomy or some other procedure. These procedures are also characterized by relatively long recovery times and are highly invasive procedures.

The currently available therapies are not condro-protective. Previously, a popular form of arthroplasty was interpositional arthroplasty with interposition of some other tissue like skin, muscle or tendon to keep inflammatory surfaces apart or excisional arthroplasty in which the joint surface and bone was removed leaving scar tissue to fill in the gap. Other forms of arthroplasty include resection(al) arthroplasty, resurfacing arthroplasty, mold arthroplasty, cup arthroplasty, silicone replacement arthroplasty, etc.

Osteotomy is a related arthroplasty procedure involving cutting of bone to improve alignment. The goal of osteotomy is to relieve pain by changing the anatomy and equalizing forces across the joint. This procedure is often used in younger, more active or heavier patients. Hip osteotomy involves removing bone from the femoral head or from the acetabulum of the hip joint and moving the bones slightly within the joint. This changes the position of the bones of the hip joint to shift the brunt of the patient's weight from damaged joint surfaces to healthier cartilage. A metal plate or pin is inserted to keep the bone in the new position.

Other approaches to treating osteoarthritis involve an analysis of loads which exist at a joint. Both cartilage and bone are living tissues that respond and adapt to the loads they experience. If a joint surface remains unloaded for appreciable periods of time the cartilage tends to soften and weaken. Further, as with most materials that experience structural loads, particularly cyclic structural loads, both bone and cartilage begin to show signs of failure at loads that are below their ultimate strength. However, cartilage and bone have some ability to repair themselves. There is also a level of load at which the skeleton will fail catastrophically. Accordingly, it has been concluded that the treatment of osteoarthritis and other conditions is severely hampered when a surgeon is not able to precisely control and prescribe the levels of joint load. Furthermore, bone healing research has shown that some mechanical stimulation can enhance the healing response and it is likely that the optimum regime for a cartilage/bone graft or construct will involve different levels of load over time, e.g. during a particular treatment schedule. Thus, there has been identified a need for devices which facilitate the control of load on a joint undergoing treatment or therapy, to thereby enable use of the joint within a healthy loading zone.

Certain other approaches to treating osteoarthritis contemplate external devices such as braces or fixators which control the motion of the bones at a joint or apply cross-loads at a joint to shift load from one side of the joint to the other. Various of these approaches have had some success in alleviating pain but suffer from lack of patient compliance or lack an ability to facilitate and support the natural motion and function of the diseased joint. Notably, the motion of bones forming a joint can be as distinctive as a finger print, and thus, each individual has his or her own unique set of problems to address. Some prior approaches to treating osteoarthritis have also been remiss in acknowledging all of the basic functions of the various structures of a joint in combination with its unique movement.

Osteoarthritis is the most common type of hip arthritis. As the protective cartilage is worn away by hip arthritis, bare bone is exposed within the joint.

Hip arthritis typically affects patients over 50 years of age. It is more common in people who are overweight, and weight loss tends to reduce the symptoms associated with hip arthritis. There is also a genetic predisposition of this condition, meaning hip arthritis tends to run in families. Other factors that can contribute to developing hip arthritis include traumatic injuries to the hip and fractures to the bone around the joint.

It has been reported that thirty-five percent of all osteoarthritis is found in the hips. In fact, it has been estimated that more than ten million American adults suffer from hip osteoarthritis and more than $6 billion is spent per year treating hip osteoarthritis. Hip osteoarthritis is particularly debilitating. Generally, it is believed that osteoarthritis of the hip is the most disabling of all joint osteoarthritis.

The most common symptoms of hip arthritis are pain with activities, limited range of motion, stiffness of the hip, walking with a limp, and decreased function, strength, activities and quality of life. Hip arthritis symptoms tend to progress as the condition worsens. Interestingly, hip arthritis symptoms do not always progress steadily with time. Often, patents report good months and bad months or symptom changes with weather accurately represent the overall progression of the condition.

Evaluation of a patient hip arthritis should begin with a physical examination and x-rays to determine which course of treatment should be followed. Weight loss is probably one of the most important, yet least commonly performed treatments. The less weight the joint has to carry, the less painful activities will be. Also, limiting certain activities may be necessary, and learning new exercise methods may be helpful. Strengthening of the muscles around the hip joint may help decrease the burden on the hip. Preventing atrophy of the muscles is an important part of maintaining functional use of the hip. Anti-inflammatory pain medications (NSAIDs) are prescription and nonprescription drugs that can help treat pain and inflammation. In more intrusive approaches, hip replacement surgery can hip resurfacing surgery have also been employed to treat hip osteoarthritis. In the most common hip replacement surgery, the cartilage is removed and a metal and plastic ball and socket hip replacement implant is placed in the hip. As an alternative to hip replacement, some patients are opting to pursue hip resurfacing surgery.

However, there is a need for a treatment modality which bridges the gap between the more conservative approaches such as weight loss, physical therapy and anti-inflammatory medicine and a decision to seek major surgical intervention. Such a treatment modality should be minimally invasive yet sufficiently effective to reduce the pain of osteoarthritis. The treatment should also be compatible with hip anatomy taking into consideration the many muscles overlaying the hip joint without hindering motion and avoiding the major arteries and nerves which are present.

The present disclosure addresses these and other needs.

### SUMMARY

Briefly and in general terms, the present disclosure is directed towards apparatus and methods for treating the hip. Various structures are presented to specifically treat osteoarthritis of the hip joint.

In one aspect, there is disclosed an apparatus for controlling a load on a human hip joint during normal gait while preserving full range of motion. The apparatus includes a first fixation assembly for attachment to a pelvis, a second fixation assembly for attachment to a femur, and a link assembly coupled to the first fixation assembly and to the second fixation assembly, the link assembly including an absorber element configured to counteract the natural compressive forces experienced by the hip joint and a pivot permitting 150° range of motion when the hip joint is in flexion and 15° range of motion when the hip joint is in extension.

In another aspect, the apparatus for controlling loads includes a first fixation assembly configured for attachment to a pelvis at a first location, a second fixation assembly configured for attachment to a femur bone at a first location, a first link assembly coupled to the first fixation assembly and to the second fixation assembly, a third fixation assembly configured for attachment to the pelvis at a second location, a fourth fixation assembly configured for attachment to a femur bone at a second location, a second link assembly coupled to the third fixation assembly and coupled to the fourth fixation assembly, wherein the link assemblies each include a compression spring configured to counteract natural compressive forces experienced by the hip joint and permit motion of the hip joint in flexion, extension, abduction, adduction and rotation.

Further, in one embodiment, the link assembly can include a biasing structure configured to counteract the natural compressive forces experienced by the hip joint and a pivot permitting 150° range of motion inflexion, 15° range of motion in extension, 30° range of motion in abduction, 10° range of motion in adduction and 20° internal and external rotation.

In a related method, a first fixation assembly is attached to a pelvis, a second fixator assembly is attached to a femur and a link assembly which provides reduction of pressure on at least a portion of the hip joint without substantially resisting full mobility of the hip joint is attached to the first and second fixator assemblies.

In another embodiment, an apparatus for controlling a load on a hip joint while preserving full range of motion comprises a first fixation assembly for attachment to a pelvis, a second fixation assembly for attachment to a femur, and a link assembly coupled to the first fixation assembly and coupled to the second fixation assembly. The link assembly includes a biasing structure configured to counteract the natural compressive forces experienced by the hip joint and a pivot permitting 150° range of motion in flexion, 15° range of motion in extension, 30° range of motion in abduction, 10° range of motion in adduction, and 20° internal and external rotation.

Other features and advantages of the present disclosure will become apparent from the following detailed description, taken in conjunction with the accompanying drawings, which illustrate, by way of example, the principles of the invention.

### BRIEF DESCRIPTION OF THE DRAWINGS

FIG. 1 is a front view, depicting a hip joint;

FIG. 2 is a partially exploded and enlarged view, depicting details of the hip joint;

FIGS. 3A and 3B are front views, depicting the arteries and nerves of the hip joint;

FIG. 4 is a perspective view, depicting angles of forces typically found in a hip joint;

FIG. 5 is a chart, depicting a force and motion relative of a hip joint during gait;

FIG. 6 is a front view, depicting a healthy hip joint and a hip joint suffering from osteoarthritis;

FIG. 7 is a front view, depicting a first embodiment of a load controlling device placed across a hip joint;

FIG. 8 is a front view, depicting a second embodiment of a load controlling device placed across a hip joint;

FIG. 9A is a front view, depicting a third embodiment of a load controlling device placed across a hip joint;

FIG. 9B is an exploded view, depicting the apparatus shown in FIG. 9A;

FIG. 10 is a side view, depicting multiple load controlling devices placed across a hip joint;

FIG. 11 is a front view, depicting a fourth embodiment of a load controlling device placed across a hip joint;

FIG. 12 is a side view, depicting a fourth embodiment of a load controlling device placed across a hip joint; and

FIG. 13 is a front view, depicting a fifth embodiment of a load controlling device placed across a hip joint.

### DETAILED DESCRIPTION OF THE PREFERRED EMBODIMENTS

Referring now to the drawings, which are provided by way of example and not limitation, the present invention is directed towards apparatus and methods for treating the hip joint. The present disclosure seeks to alleviate pain associated with the function of diseased or malaligned members forming the hip joint. Whereas the present disclosure is particularly suited to address issues associated with osteoarthritis, the energy manipulation accomplished by the disclosed apparatus and methods lends itself well to broader applications.

In one particular aspect, the present disclosure seeks to permit and complement the unique articulating motion of the members defining a hip joint of a patient while simultaneously manipulating energy being experienced by both cartilage and osseous tissue (cancellous and cortical bone). Approaches involving varying energy absorption and transfer during the pivoting of the joint and selecting a geometry for the energy absorption assembly to provide necessary flexibility are implemented into various embodiments of the present disclosure. Certain of the embodiments include geometry which accomplishes variable energy absorption designed to minimize and complement the dampening effect and energy absorption provided by the anatomy of the body, such as that found at a hip joint. In certain specific applications, distraction is employed in the energy manipulation approach.

In one particular approach, a bending spring assembly is contemplated to manipulate or absorb forces between body parts. Thus, an assembly utilizing an element or elements which respond to bending or changes in elongation may be desirable to treat afflictions such as osteoarthritis. Certain of the assemblies can incorporate features which insure correct device alignment as the member transitions between compressed and uncompressed states.

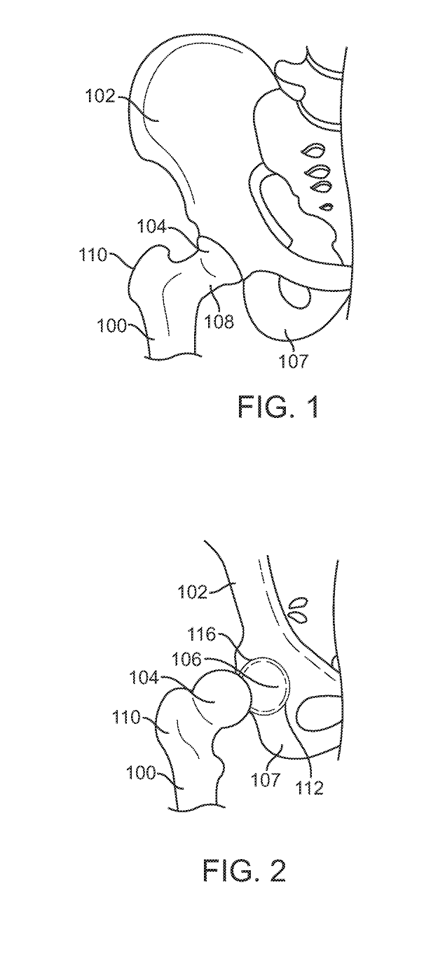

With specific reference to FIGS. 1-3C, certain features of typical hip anatomy are presented. The hip joint is a ball-and-socket joint. This arrangement gives the hip a large amount of motion needed for daily activities like running, walking, sitting, and climbing stairs. The deepest layer of the hip includes the bones and the joints. The next layer is made up of the ligaments of the joint capsule. Tendons and the muscles overlay the ligaments and joint capsule.

The structures of the hip can be divided into several categories. These include bones and joints, ligaments and tendons, muscles and blood vessels. The bones of the hip are the femur (the thighbone) 100 and the pelvis 102. The top end of the femur is shaped like a ball. This ball is called the femoral head 104. The femoral head 104 fits into a round socket on the side of the pelvis referred to as the acetabulum 106 (FIG. 2). Structure extending below and lateral to the acetabulum 106 is the portion of the pelvis called the ischium 107.

The femoral head 104 is attached to the rest of the femur 100 by a short section of bone called the femoral neck 108. A large bump juts outward from the top of the femur 100, next to the femoral neck 108. This bump, or the greater trochanter 110, can be flat along the side of your hip. Large muscles connect to the greater trochanter 110.

Articular cartilage 112 is the material that covers the ends of the bones of any joint (FIG. 2). Articular cartilage is about one-quarter of an inch thick in the large, weight-bearing joints like the hip. Articular cartilage has a rubbery consistency and is slippery, which allows the joint surfaces to slide against one another without causing damage. The function of articular cartilage is to absorb shock and provide an extremely smooth surface to make motion easier. In the hip, articular cartilage covers the end of the femur 100 and the socket portion of the acetabulum 106 in the pelvis. The cartilage is especially thick in the back part of the socket, as this is where most of the force occurs during walking and running.

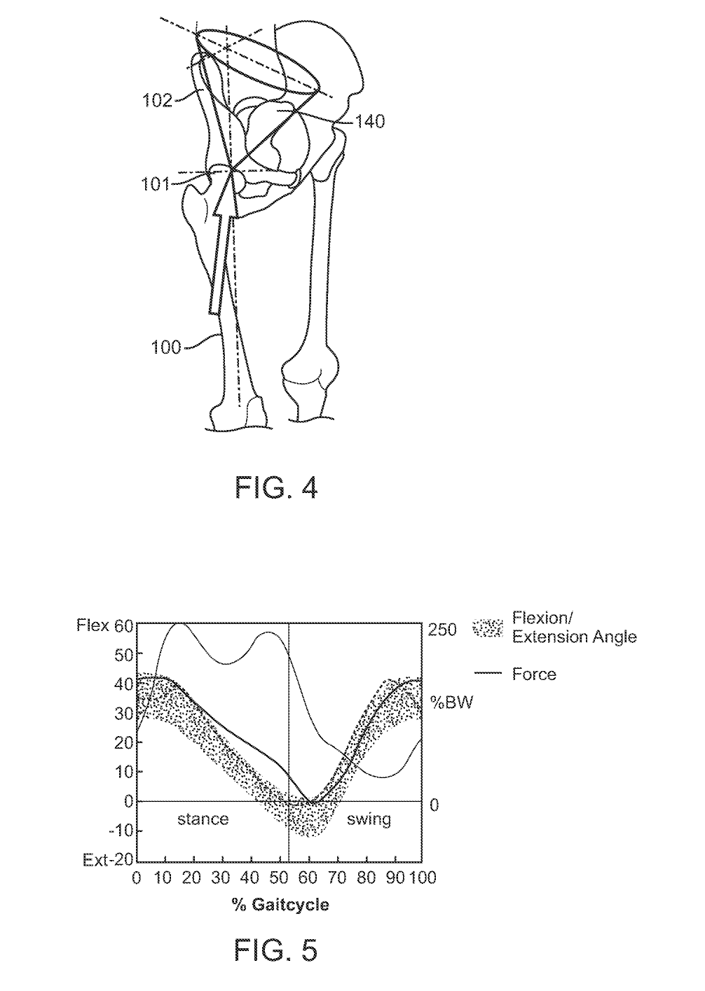

A cone 140 depicted in FIG. 4 exhibits the direction of the majority of forces applied to the hip socket during the gait cycle. A goal of the load controlling apparatus for the hip is to off-load some of these forces while maintaining as much natural motion as is possible. It has been found that moderate cyclic loading on the hip joint is beneficial and necessary to biological health.

There are several ligaments in the hip. Ligaments are soft tissue structures that connect bones to bones. A joint capsule is a watertight sac that surrounds a joint. In the hip, the joint capsule is formed by a group of three ligaments that connect the femoral head to the acetabulum. These ligaments are the main source of stability for the hip and help hold the hip in place. A special type of ligament forms a unique structure inside the hip called the labrum 116 (FIG. 2). The labrum is attached almost completely around the edge of the acetabulum 106. The shape and the way the labrum 116 is attached create a deeper cup for the acetabulum socket. This small rim of cartilage can be injured and cause pain and clicking in the hip.

The hip is surrounded by thick muscles. The gluteals make up the muscles of the buttocks on the back of the hip. The inner thigh is formed by the adductor muscles. The main action of the adductors is to pull the leg inward toward the other leg. The muscles that flex the hip are in front of the hip joint. These include the iliopsoas muscle. This deep muscle begins in the low back and pelvis and connects on the inside edge of the upper femur. Another large hip flexor is the rectus femoris. The rectus femoris is one of the quadriceps muscles, the largest group of muscles on the front of the thigh. Smaller muscles going from the pelvis to the hip help to stabilize and rotate the hip. The load controlling apparatus can be located beneath the muscles and ligaments of the hip or can be positioned between the muscles and tendons.

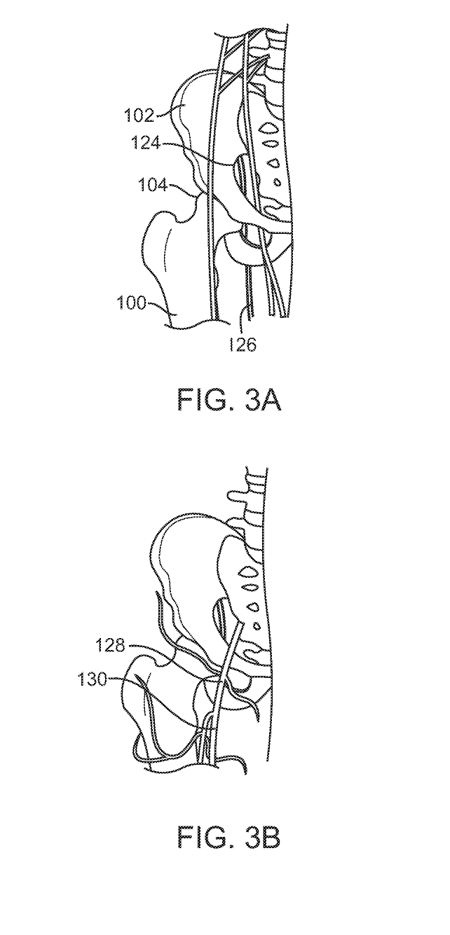

All of the nerves that travel down the thigh pass by the hip (See FIG. 3A). The main nerves are the femoral nerve 124 in front and the sciatic nerve 126 in back of the hip. A smaller nerve, called the obturator nerve (not shown), also goes to the hip. These nerves carry the signals from the brain to the muscles that move the hip. Traveling along with the nerves are the large vessel that supply the lower limb with blood (See FIG. 3B). The large femoral artery 128 begins deep within the pelvis. It passes by the front of the hip area and goes down toward the inner edge of the knee. The femoral artery has a deep branch, called the profunda femoris 130. The profunda femoris sends two vessels that go through the hip joint capsule. These vessels are the main blood supply for the femoral head. Other small vessels form within the pelvis and supply the back portion of the buttocks and hip.

The hip joint has the greatest range of movement of any joint, second only to the shoulder. The full range of motion of the hip is much larger than used in daily routine activities. For example, in flexion and extension this maximum range of motion is 150° and 15° respectively while during normal gait, there is typically only 45° of flexion and 10° of extension. For abduction/adduction, the maximum range of motion is 45°/30° whereas during normal gait, the range of motion is closer to 7°/10°. Further, as to internal/external rotation, the maximum range of motion can be 60°/60° and only 4°/3° during normal gait. Interestingly, however, in patients suffering from hip osteoarthritis normal gate changes and the range of motion increases in some motions and decreases in other motions. That is, flexion/extension or an osteoarthritic hip during walking has been observed to be 25°/18°, abduction/adduction at 4°/7° and internal/external rotation at 10°/7°.

Referring now to FIG. 5, a force-motion relationship of a hip joint during gait is presented. Of particular interest to treatment, it is noted that peaks in load occur between 40° and 10° flexion during the stance portion of the gate cycle. It is further noted that vertical forces dominate in the hip joint. It has also been observed that forces of 2.4 times body weight are common during normal walking Higher forces of up to about 9 times body weight are observed in the hip when running, stumbling or walking down stairs. Lower forces exist when sitting or standing.

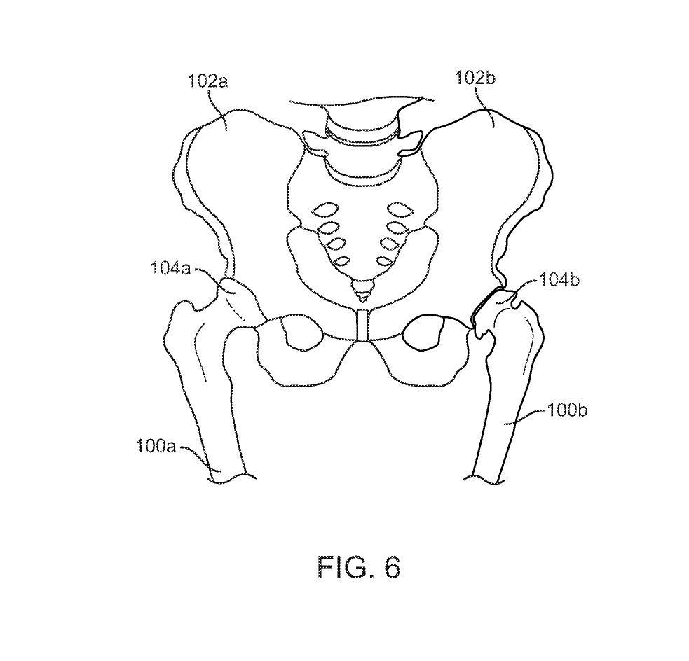

As shown in FIG. 6, showing a healthy hip 102a and an osteoarthritic hip 102b, osteoarthritis can be exhibited as osteophytes and deteriorated cartilage. Often, changes in the femur head 104 and the acetabulum 106 mirror each other or are similar. Various overlapping steps in osteoarthritis include initial articular cartilage degeneration, osteophyte formation in areas of low or no load causing decreases in range of motion and the flattening of the femoral head. Eburnation where bared bone and cartilage are worn away occurs as does necrosis, sclerosis and cyst formation.

Aside from ostephyte formation, osteoarthritis develops primarily in regions of high load. As shown by the schematic cone 140 in FIG. 4, the majority of forces in the hip joint are through anterior, superior and medial regions of the femoral head 104. Thus, devices for manipulating such loads are contemplated to fight the degeneration of the hip joint.

The load manipulation devices of the present disclosure are contemplated to be placed across a hip joint. Fixation structures for fixing the load manipulation structures to the bone may be attached, for example, to the femur and pelvis. These are linked together with an absorber, the linking structure being configured to accommodate natural motions of the hip joint. The absorber is designed to absorb or off-load some portion of the load normally carried by the hip joint. The device itself is positioned such that the resultant force/unloading vector corresponds to a desired direction of off-loading. In particular, unloading is contemplated to occur in directions relating to the force cone 140. It is also contemplated that multiple load manipulation devices can be placed at various locations across the hip joint to thereby balance out and/or decrease moment forces being accommodated.

In various approaches, on the femur side of a hip joint, the load manipulation devices can be attached at inferior and superior positions, to the greater trochanter, the lesser trochanter, at ostephytes on the femur, or along a bend in the neck of the femur. The device can also be affixed all around the neck and head of the femur including the base and underside of the femur head. Also, it is contemplated that on the femur side, the fixation point be beneath, within, above or in place of muscle. On the pelvis side, fixation of the absorber can be at the top or bottom of the acetabulum, along a ridge defining the acetabulum or along surrounding areas. Additionally, various surfaces on and around the ischium are also contemplated for fixation. Muscle may need to be displaced to gain sufficient access to the ischium to effect a proper fixation.

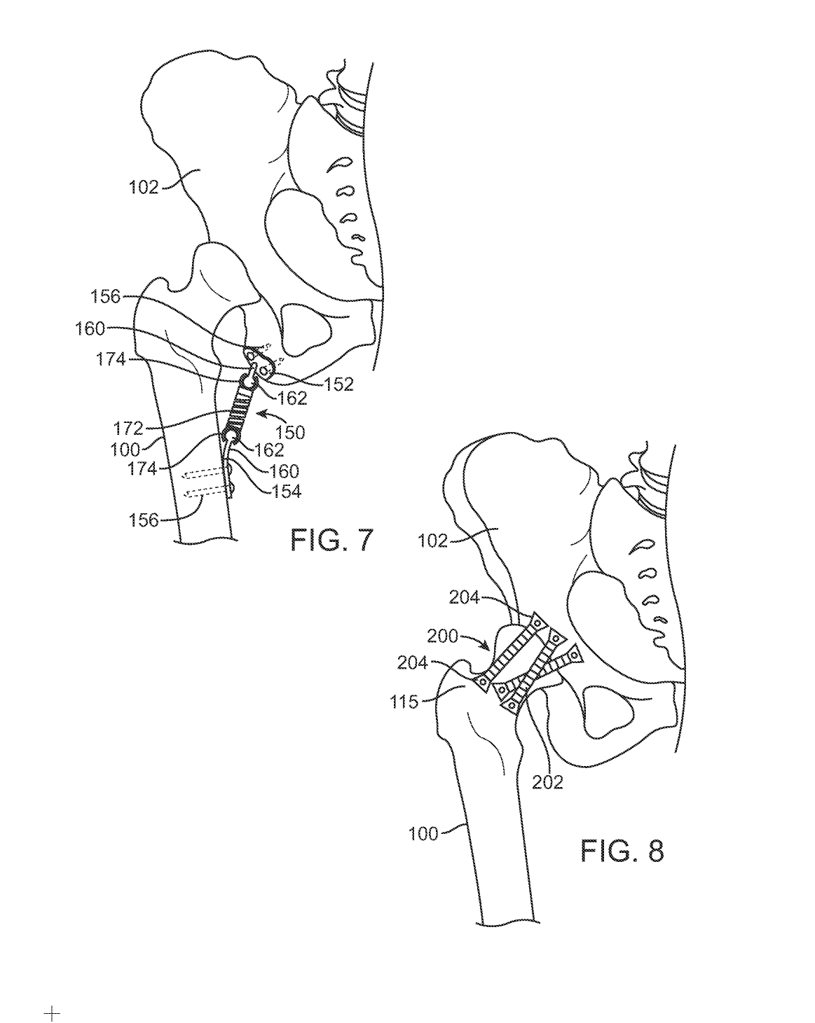

With reference now to FIG. 7, there is shown one embodiment of an absorber or load manipulation assembly 150 implanted across a hip joint. In this embodiment, the absorber assembly 150 is designed to control a load on a hip joint during normal gait while preserving as close to natural motion of the members of the hip joint as possible. In one example, the load manipulation assembly 150 includes a first fixation assembly 152 for attachment to the pelvis 102 and a second fixation assembly 154 for attachment to the femur 100. The first and second fixation assemblies 152, 154 can be plates that are specifically shaped or contoured to overlay mounting sites of the pelvis and femur. In one particular aspect, the first fixation assembly 152 can be configured to attach to the pelvis, for example, on the ischial tuberosity of the pelvis. The second fixation assembly 154 is fixed on a medical side of the femur at a location which can be on or below the lesser trochanter. Fixation members such as locking screws 156 can be employed to secure the fixation assemblies 152, 154 in place. In one example, the load manipulation assembly 150 is positioned superior to the major nerves and arteries including the femoral nerve and the femoral artery.

Projecting from each of the fixation assemblies 152, 154 and configured to extend away from the pelvis 102 and femur 100 are shafts 160. The shafts 160 have terminal ends configured with a ball 162.

An absorber 170 is configured between the fixator assemblies 152, 154. The absorber 170 includes a central spring 172 and a pair of spaced ends forming a socket 174. The sockets 174 are sized and shaped to receive the ball structure formed on the ends of the shafts 160. This ball in socket arrangement is designed to permit the full range of motion of a healthy hip joint while the spring 172 absorbs a percentage of the forces on the hip joint during normal hip action. In an alternative embodiment, the ball and socket arrangements can be reversed with the balls arranged on the absorber and the sockets arranged on the fixation assemblies.

The spring 172 can be supported on a rigid telescoping piston arrangement which allows the absorber to extend and compress without bending. Although the absorber 170 is illustrated with a single spring 172, multiple springs may be used to provide load absorption. Additional examples of absorber designs which may be used in place of the absorber 170 are shown in U.S. Published Patent Application No. 2008/0275558, which is incorporated herein by reference in its entirety.

In order to accommodate the natural motion of the hip joint in abduction, the absorber is extendable from a fully compressed length to a fully extended length. When the absorber is positioned as in the example of FIG. 7, the fully extended absorber length is between 150% and 190% of the compressed length. The load manipulating assembly 150 is shown with two ball and socket joints and a telescoping absorber, providing seven degrees of freedom for maximum flexibility. Other embodiments of the assembly may include a pivot joint in place of one or more of the ball and socket joints and/or a flexible absorber.

To implant the load manipulating assemblies of the present invention, conventional surgical or minimally invasive approaches can be taken to gain access to a body joint or other anatomy requiring attention. Arthroscopic approaches are contemplated when reasonable to both implant the energy manipulation assembly as well as to accomplish adjusting an implanted assembly. Biologically inert materials of various kinds can be employed in constructing the energy manipulation assemblies of the present invention. The materials can include titanium or titanium alloy, cobalt chromium alloy, ceramic, high strength plastic such as polyetheretherketone (PEEK), PEEK composites including carbon fibre reinforced and graphite reinforced PEEKs or other durable materials. Combinations of materials can also be used to maximize the properties of materials for different parts of the device. At the wear surfaces, the material may include a combination of metal-on-poly, metal-on-metal, metal-on-ceramic or other combinations to minimize wear.

In a related approach (See FIG. 8), an energy manipulation assembly 200 can be embodied in an elastomeric absorber element 202. The elastomeric absorber element 202 stiffens in compression and is capable of both elongation and bending. Opposing terminal end portions 204 flare outwardly from a center section of the elastomeric absorber element 202. The opposing end portions 208 include through holes sized and shaped to receive fixation members such as screws. Thus, the opposing end portion 208 can be affixed to bone so that the device spans a joint. Here, one end of the device is attached to the pelvis 102 and the other end is attached to the femur 100.

In one embodiment, the absorber element 202 can be comprised of silicone, silicone ePTFE, or other elastomeric materials which permit lengthening but resist compression beyond a given amount. Soft and hard segments can be disbursed along the elastomeric absorber to provide the desired compression and lengthening. On compression beyond a neutral position of the absorber, the hard segments provide resistance to compression and restrains the soft sections. The lengthening as well as flexibility of the absorber element is set such that the full or nearly full range of motion of a hip joint is maintained. Although the absorber elements 202 are shown arranged on the anterior of the hip joint, they can also be arranged on the posterior, medial and lateral sides of the hip joint. They can be a converging or diverging angles with respect to one another or crossing arrangements as shown in FIG. 8.

As with each of the disclosed embodiments, a plurality of energy manipulation devices can be positioned across a hip joint. For this and other embodiments, the fixation points of the device are contemplated to be outside of the hip capsule and the absorber is positioned along natural planes and lines of the hip ligaments and away from major arteries and nerves. On the femur side, fixation of terminal ends 204 of the energy manipulation device 200 can for example, be configured to be placed along the greater trochanter 115 of the femur 100.

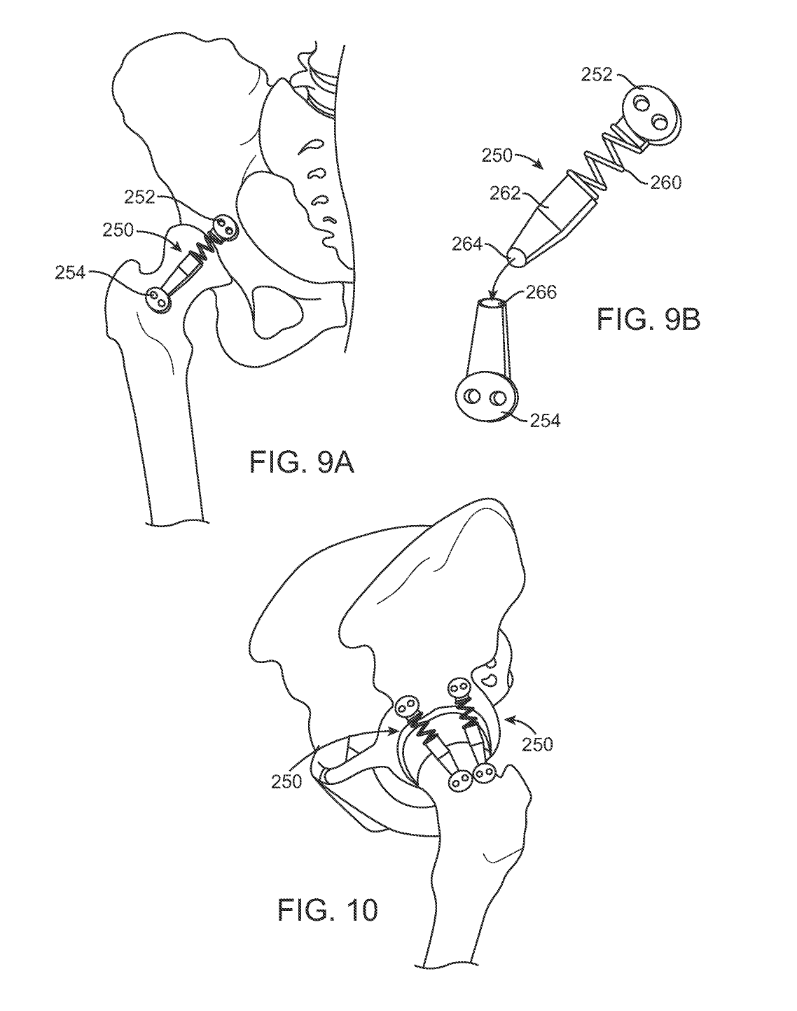

Yet another related approach is shown in FIGS. 9A-10. In this approach, the energy manipulation assembly 250 includes first 252 and second 254 terminal ends adapted for fixation to bones on opposite sides of the hip joint. As stated above, the fixation points can be on various surfaces of the pelvis and femur as well as through or about muscles while avoiding arteries and nerves. The terminal ends 252, 254 are characterized by flattened sections having through holes formed therein and sized to receive fixation screws. The mid-section of the assembly includes a spring 260 extending from the first fixation end 252. Although the spring 260 is shown as a simple coil spring, the spring may take on different shapes and may include a central piston structure for stability similar to the absorber of FIG. 7. The end of the spring 200 opposite the fixation end 252 is provided with an arm 262 terminating in a ball structure 264.

The ball structure 264 of the energy manipulation assembly 250 is rotatably engaged within a socket 266 projecting from the second fixation structure 254. In this way, it is contemplated that full mobility of the hip joint is preserved while the spring off-loads and/or manipulates forces bearing on the hip joint. As shown in FIG. 10, various of these load manipulation assemblies 250 can be positioned across the hip joint to accomplish desired treatment. Thus, absorber assemblies can be positioned strategically to provide a necessary effect depending on the patient anatomy and the areas of deterioration of the joint. For example, a single absorber can be placed superiorly or separate absorbers can be placed in both inferior and superior locations. In one specific approach, separate devices can be placed at anterior (double at 70° anterior) and posterior (80° posterior) positions.

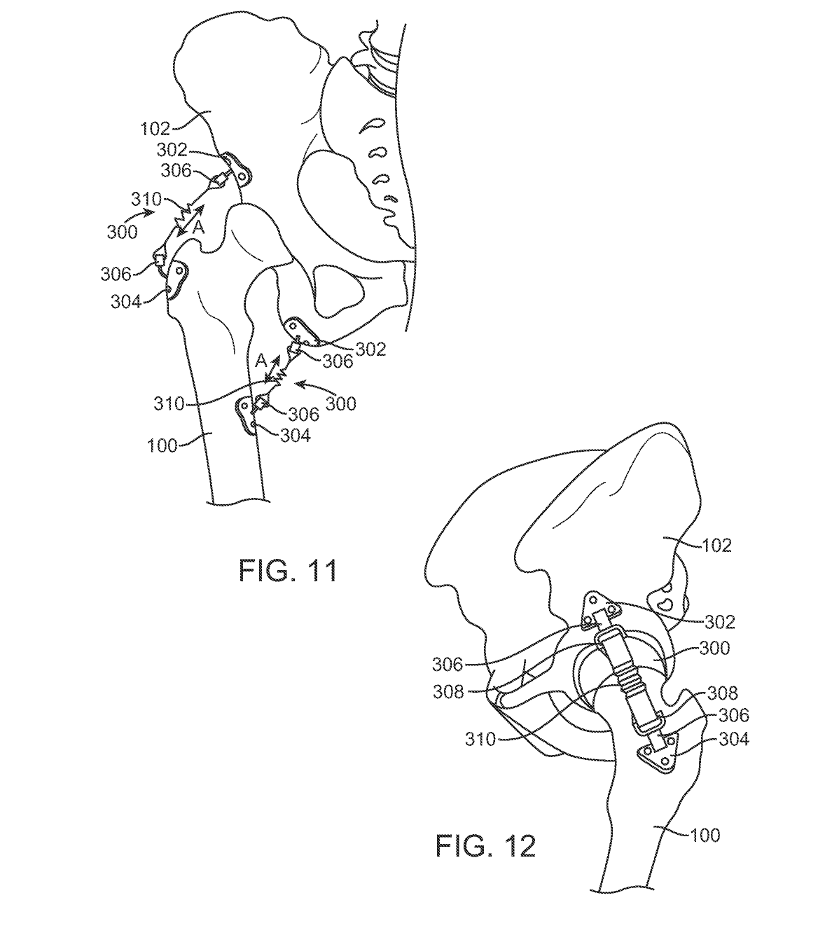

FIGS. 11 and 12 illustrate still another approach to an energy absorber assembly 300. The assembly again includes fixation structures 302, 304 adapted for attachment to the various contemplated areas of the pelvis 102 and femur 100, respectively. Also, it is again contemplated that a single assembly or multiple assemblies 300 can be configured across a hip joint. In this embodiment, the fixation structures 302, 304 each include an arm 306 terminating with a loop 308 (See FIG. 12). An absorber in the form of a spring-like absorber mid-section 310 is configured to engage the loops 308. The absorber 310 is shown schematically in FIGS. 11 and 12 as a spring. The absorber 310 applies a force in the direction of the arrows A in FIG. 11, while apply on force in a direction opposite to the arrows. In this manner. The absorber 310 provides unloading of the hip joint during the portions of the gait cycle with the highest forces on the joint. However, the absorber 310 may provide less or no unloading at other positions, such as a seated position.

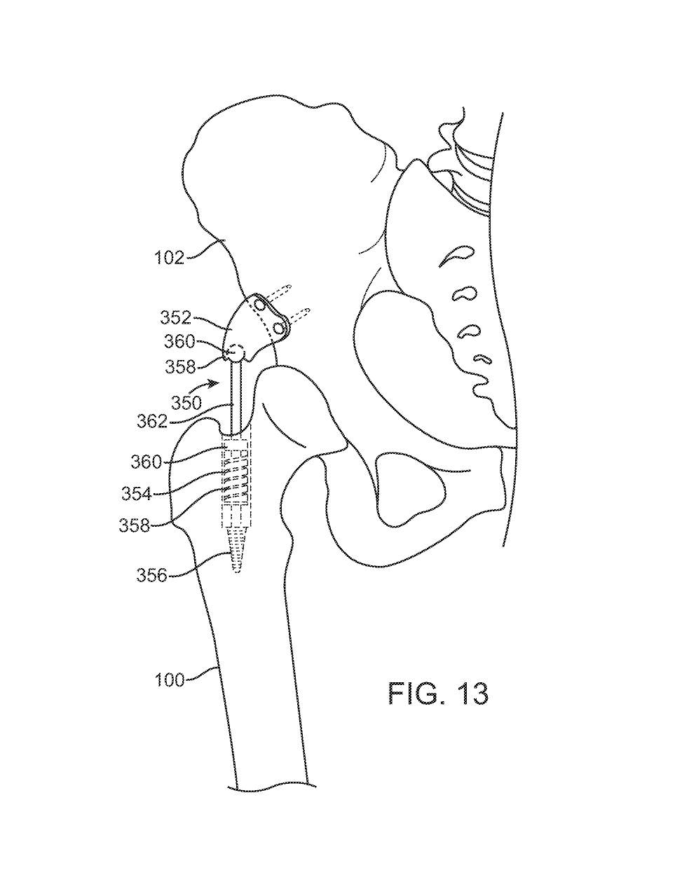

Turning now to FIG. 13, there is shown a load manipulation assembly 350 characterized by a first fixation structure 352 assuming the profile of a plate and a second fixator structure 354 extending within femur. Here, the second fixation structure 354 extends through a hole drilled in the femur from the greater trochanter notch and extending substantially axially within the femur. However, it is to be recognized that in this example as well as previously contemplated approaches, such internal-bore fixation structures can be at either or both ends of an energy absorption assembly. Thus, as shown, the first fixation structure 352 includes a plate configured to conform to bone and has a plurality of through holes for receiving fixation screws. The second fixation structure 354 includes a longitudinally extending member having fixation members, such as threads 356 extending at least a portion of its length. Such threads facilitate a secure connection bone and may be used alone or in conjunction with cement, screws or other members.

An absorber 358 is positioned within the bore for the second fixation structure 354 to provide an unloading force to the hip joint by one or more springs.

In this approach, the first fixation structure 352 also includes a socket structure 358 for receiving a ball end 360 of a projection 362 extending from the second fixation structure 354. The ball-in-socket arrangement thus preserves full motion of the hip joint while the assembly accomplishes needed load manipulation. The projection 362 or shaft of the first fixation structure also functions as a piston of the absorber 358. A collar 360 on the piston 362 provides a biasing surface for the free end of the spring. The piston 362 can slide outward to allow normal range of motion of the hip joint.

It is to be borne in mind that each of the disclosed various structures can be interchangeable with or substituted for other structures. Thus, aspects of each of the assemblies can be employed across approaches. Moreover, the various manners of engaging energy absorbing structure with attachment structure and attachment structures to body anatomy can be utilized in each approach. Also, one or more of the various disclosed assemblies can be placed near a treatment site and at various angles with respect thereto. Pressure sensing and drug delivery approaches can also be implemented in each of the various disclosed embodiments.

Certain components of most embodiments of the present invention can be designed for easy removal and, if necessary replacement while others are intended for permanent fixation. The devices can also be implanted encased within a sheath. The permanent components are fixation components which have bony ingrowth promoting surfaces and are responsible for fixation of the system to the skeletal structure. The removable components include the mobile elements of the system such as the link members and/or the pivots or ball joints.

The advantages of this feature of the system include the ability to exchange key components of the system due to device failure, patient condition change or newer improved systems being available. Additionally if the patient subsequently requires further surgery the links may be removed to facilitate the additional procedure.

Further, certain of the contemplated mechanisms can be made to be completely disengaged mechanically and then brought into action under various conditions and during certain phases of the gait cycle. This discontinuous functionality—and the ability to tune that functionality to a particular patient's gait or pain is consequently a feature of the present invention.

Location of the permanent fixation components is important to fixation strength, ability to complete subsequent procedures, and location of pivots or ball joints. The fixation strength of the system, and therefore load bearing capacity, is dependent on the integrity of the bone onto which the plate is fixed. To ensure strong fixation, in one embodiment, the fixation components span along the cortical bone and cancellous (or trabecular) bone. The plate can reside on the femoral shaft and extend down onto the trabecular bone on the end of the femur. Also, the system may utilize fixation on two cortical surfaces using through pins or bicortical screws.

A common joint procedure is joint replacement as previously described. The procedure of replacing a diseased joint includes resection of the surfaces of the joint and replacement with synthetic materials. To enable implantation of the energy absorbing system without impacting the potential to complete subsequent procedures (e.g., joint replacement) the permanent fixation components in a preferred embodiment are positioned at a location that does not compromise the total joint zone.

Many articulating joints are not simply pivot joints but involve complex multi-axis rotation and translation movements. To achieve its intended purpose, the energy absorber must accommodate these movements but also absorb and transfer energy during the required range of motion. To do so the joints on the device may be either located at points on the bones of least motion, or the joint mechanism must incorporate motion beyond simple uni-axial rotation or a combination of both.

Further, the fixation components can be positioned such that they orientate the attached device joint locations to preferred locations described by minimal or known motion characteristics. The device joint locations may be finely adjusted within a defined region on the fixation component to further optimize the device joint location. The device joint mechanism can also be configured to accommodate the positional changes and therefore can be placed on any distal point on the fixation component.

Therefore, the present invention provides a number of ways to treat body tissues and in particular, to absorb energy or manipulate forces to reduce pain. The disclosed devices can be used throughout the body but have clear applications to articulating body structures such as joints.

Thus, it will be apparent from the foregoing that, while particular forms of the invention have been illustrated and described, various modifications can be made without parting from the spirit and scope of the invention.

We claim:

1. An apparatus for controlling a load on a human hip joint during normal gait while preserving motion, comprising:
a first fixation assembly for attachment to a pelvis;
a second fixation assembly for attachment to a femur; and
a link assembly coupled to the first fixation assembly and to the second fixation assembly, the link assembly including an absorber element configured to counteract the natural compressive forces experienced by the hip joint and a pivot permitting 150° range of motion when the hip joint is in flexion and 15° range of motion when the hip joint is in extension.

2. The apparatus of claim 1, wherein the first and second fixation assemblies are each angularly displaceable relative to the link assembly.

3. The apparatus of claim 1, wherein the link assembly is connected to at least one of the first and second fixation assemblies by a universal joint.

4. The apparatus of claim 3, wherein the universal joint is a ball joint.

5. The apparatus of claim 1, wherein the first fixation assembly is configured for attachment to the ischium of the pelvis.

6. The apparatus of claim 1, further comprising a third and a fourth fixation assembly for connecting a second link assembly across the hip joint.

7. The apparatus of claim 6, wherein the first fixation assembly is configured for attachment to the ischium of the pelvis and wherein the third fixation assembly is configured for attachment to the ilium of the pelvis.

8. The apparatus of claim 1, wherein the link assembly is configured as a piston and arbor assembly configured to unload the hip joint by compression of the absorber element, and wherein the piston and arbor assembly extends to allow near natural motion of the hip joint.

9. The apparatus of claim 1, wherein the absorber element is a compression spring.

10. The apparatus of claim 1, wherein the entire apparatus is configured to be implanted outside of the hip joint capsule.

11. The apparatus of claim 1, wherein the absorber element is an elastomeric element which stiffens in compression, is bendable and capable of elongation.

12. The apparatus of claim 1, wherein at least a portion of the link assembly is positioned within the bone of the femur or pelvis.

13. A system for controlling a load on a human hip joint while preserving motion, comprising:
a first fixation assembly configured for attachment to a pelvis at a first location;
a second fixation assembly configured for attachment to a femur bone at a first location;
a first link assembly coupled to the first fixation assembly and to the second fixation assembly;
a third fixation assembly configured for attachment to the pelvis at a second location;
a fourth fixation assembly configured for attachment to a femur bone at a second location;
a second link assembly coupled to the third fixation assembly and coupled to the fourth fixation assembly;
wherein the link assemblies each include a compression spring configured to counteract natural compressive forces experienced by the hip joint and permit motion of the hip joint in flexion, extension, abduction, adduction and rotation.

14. The apparatus of claim 13, wherein the first and second locations are on substantially opposite sides of the hip socket.

15. The apparatus of claim 13, wherein the first and second pivots provide at least two degrees of rotational freedom.

16. The apparatus of claim 15, wherein the link assemblies each include a pivot joint.

17. The apparatus of claim 16, wherein the first and second pivots are universal joints.

18. The apparatus of claim 13, wherein the first and second fixation assemblies are each angularly displaceable relative to the link assembly.

19. The apparatus of claim 13, wherein the first fixation assembly is configured for attachment to the ischium of the pelvis.

20. The apparatus of claim 13, further comprising a third and a fourth fixation assembly for connecting a second link assembly across the hip joint.

21. The apparatus of claim 20, wherein the first fixation assembly is configured for attachment to the ischium of the pelvis and wherein the third fixation assembly is configured for attachment to the ilium of the pelvis.

22. The apparatus of claim 13, wherein the link assembly is configured as a piston and arbor assembly configured to unload the hip joint by compression of the compression element, and wherein the piston and arbor assembly extends to allow near natural motion of the hip joint.

23. The apparatus of claim 13, wherein the compression element is a compression spring.

24. The apparatus of claim 13, wherein the entire apparatus is configured to be implanted outside of the hip joint capsule.

25. The apparatus of claim 13, wherein the compression element is an elastomeric element which stiffens in compression, is bendable and capable of elongation.

26. The apparatus of claim 13, wherein at least a portion of the link assembly is positioned within the bone of the femur or pelvis.

27. An apparatus for controlling a load on a hip joint while preserving full range of motion, comprising:
a first fixation assembly for attachment to a pelvis;
a second fixation assembly for attachment to a femur; and
a link assembly coupled to the first fixation assembly and coupled to the second fixation assembly, the link assembly including a biasing structure configured to counteract the natural compressive forces experienced by the hip joint and a pivot permitting 150° range of motion in flexion, 15° range of motion in extension, 30° range of motion in abduction, 10° range of motion in adduction, and 20° internal and external rotation.

28. The apparatus of claim 27, wherein the first and second pivots are universal joints.

29. The apparatus of claim 27, wherein the link assembly further comprises a mechanism for controlling loads between the pelvis and femur only during normal gait.

30. A method for controlling a load on a hip joint while preserving full range of motion, comprising:
using an apparatus for controlling the load on the hip joint to treat arthritic conditions affecting the hip joint, the apparatus comprising:
a first fixation assembly for attachment to the pelvis;
a second fixation assembly for attachment to the femur; and
a link assembly coupled to the first fixation assembly and coupled to the second fixation assembly and configured to span anatomy affected by arthritic conditions, said apparatus provides reduction of pressure on at least a portion of the hip joint without substantially resisting an angular displacement associated with relatively full mobility of the pelvis and femur of the hip joint to thereby treat arthritic conditions affecting the joint;
attaching the first fixation assembly to the pelvis:
attaching the second fixation assembly to the femur; and
configuring the link assembly to control loads occurring in the hip joint only during normal gait.

31. The method according to claim 30, wherein normal gait includes a flexion range of motion 45°, an extension range of motion 10°, an abduction range of motion 7°, an adduction range of motion 10°, for internal rotation 4° and external rotation 3°.

32. The method of claim 30, wherein the first and second fixation assemblies are each angularly displaceable relative to the link assembly.

33. The method of claim 30, wherein the link assembly is connected to at least one of the first and second fixation assemblies by a universal joint.

34. The method of claim 33, wherein the universal joint is a ball joint.

35. The method of claim 30, further comprising attaching the first fixation assembly to the ischium of the pelvis.

36. The method of claim 30, wherein the apparatus further comprises a third and a fourth fixation assembly for connecting a second link assembly across the hip joint.

37. The method of claim 36, further comprising attaching the first fixation assembly to the ischium of the pelvis and attaching the third fixation assembly to the ilium of the pelvis.

38. The method of claim 30, wherein the link assembly is configured as a piston and arbor assembly configured to unload the hip joint by compression and wherein the piston and arbor assembly extends to allow near natural motion of the hip joint.

39. The method of claim 30, wherein the link assembly includes a compression spring.

40. The method of claim 30, further comprising implanting the entire apparatus outside of the hip joint capsule.

41. The method of claim 30, wherein the link assembly includes an elastomeric element which stiffens in compression, is bendable and capable of elongation.

42. The method of claim 30, further comprising positioning at least a portion of the link assembly within the bone of the femur or pelvis.

43. An apparatus for controlling the load on the hip joint to treat a condition affecting the hip joint, the apparatus comprising:
a first fixation assembly for attachment to the pelvis;
a second fixation assembly for attachment to the femur; and
a link assembly coupled to the first fixation assembly and coupled to the second fixation assembly and configured to span anatomy affected by the condition, said apparatus provides reduction of pressure on at least a portion of the hip joint without substantially resisting an angular displacement associated with relatively full mobility of the pelvis and femur of the hip joint to thereby treat the condition affecting the joint, wherein the link assembly includes at least one spring configured to control loads occurring in the hip joint during normal gait.
